(19) United States
(12) Patent Application Publication
(10) Pub. No.: US 2024/0001030 A1
(43) Pub. Date: Jan. 4, 2024

(54) VENOUS NEEDLE MONITORING DEVICE

(71) Applicant: Quanta Dialysis Technologies Limited, Warwick (GB)

(72) Inventor: Clive BUCKBERRY, Warwickshire (GB)

(21) Appl. No.: 18/468,441

(22) Filed: Sep. 15, 2023

Related U.S. Application Data
(63) Continuation of application No. PCT/GB2022/050669, filed on Mar. 16, 2022.

(30) Foreign Application Priority Data
Mar. 16, 2021 (GB) 2103628.0

(51) Int. Cl. A61M 5/158

(52) U.S. Cl. CPC A61M 5/158; A61M 2005/1588; A61M 2230/04; A61M 2230/50; A61M 2205/18

(57) ABSTRACT

A venous needle monitoring device. An apparatus and system for monitoring for the presence of a needle or cannula to determine if it becomes dislodged from a patient. A venous needle monitoring device comprising: a wearable device; at least one temperature sensor; at least one heart rate sensor; a controller with a transmitter. The at least one temperature sensor and at least one heart rate sensor are disposed on the wearable device. The controller and transmitter are located within the wearable device. The at least one temperature sensor and at least one heart rate sensor are in communication with the controller. The controller generates an alarm signal when a threshold value is reached based upon a temperature value measured by the at least one temperature sensor and a heart rate value measured by the at least one heart rate sensor. Wherein the transmitter is suitably disposed for transmitting the alarm signal to one or more associated medical devices.

### CROSS-REFERENCE TO RELATED APPLICATIONS

This application is a continuation of International Patent Application No. PCT/GB2022/050669, filed Mar. 16, 2022, which claims the benefit of foreign priority to United Kingdom Patent Application No. 2103628.0, filed Mar. 16, 2021, with the entire contents of each of these applications hereby incorporated herein by reference.

### TECHNICAL FIELD

The present disclosure relates to a venous needle monitoring device. Particularly, but not exclusively, the disclosure relates to an apparatus and system for monitoring for the presence of a needle or cannula to determine if it becomes dislodged from a patient.

### BACKGROUND

In a number of medical fields it is important to have access to the vascular access system by piercing the skin, for example in drug infusion or blood processing treatments like dialysis.

Several devices have been proposed for monitoring a venous needle. For example to detect if a needle becomes dislodged from a patient. Such devices may use ultrasonic sensors for monitoring a needle. An example of which is the applicants' WO2010146372A2 which discloses a vascular access monitoring device with an ultrasonic transmitter and receiver for monitoring for needle dislodgement.

The present invention provides an alternative venous needle monitoring device.

### SUMMARY

In some aspects of the disclosure, a venous needle monitoring device is provided. Accordingly, such a device can include a user wearable device including any of: an open ring, a closed ring, a band, a strap, a watch, a clip, or a patch, at least one temperature sensor, at least one heart rate sensor, a controller; and a transmitter. To this end, the wearable device can also be referred to as a wearable electronic device. The at least one temperature sensor and at least one heart rate sensor can be arranged on the wearable device so as to be able to sense at least temperature and heart rate of the user, and the controller and transmitter can be located on or within the wearable device. The at least one temperature sensor and at least one heart rate sensor can be in communication with the controller, the controller can be configured to generate an alarm signal when a threshold value is reached based upon a temperature value measured by the at least one temperature sensor and a heart rate value measured by the at least one heart rate sensor. The transmitter can be configured and arranged for transmitting the alarm signal to one or more associated medical devices.

Such aspects may include one and/or another of (and in some aspects, a plurality of, a majority of, substantially all of, and all of) the following features, functions, functionality, structure, steps, and clarifications, leading to yet further aspects:
the controller can include a memory;
the controller can be configured to measure heart rate variability using the at least one heart rate sensor, where the threshold value can be further based upon the measured heart rate variability;
the temperature sensor and/or at least one heart rate sensor can be located on a face of the wearable device;
at least one blood oxygen sensor, wherein the at least one blood oxygen sensor is arranged on the wearable device so as to be able to sense the blood oxygen saturation levels, SpO2, and/or heart rate of the user, and wherein the blood oxygen sensor is in communication with the controller;
the blood oxygen sensor is at least one of a transmissive pulse oximetry sensor or a reflectance pulse oximetry sensor;
a vibration device suitable for generating a vibration alarm;
the heart rate sensor can include an infra-red heart rate sensor with at least one photodiode and at least one LED;
the at least one photodiode and at least one LED can be separate components and can be arranged in separate locations on the wearable device, suitably for allowing transmission photoplethysmograthy when the wearable device is worn;
the at least one photodiode can be arranged adjacent the at least one LED on the wearable device suitably for allowing reflectance photoplethysmograthy when the wearable device is worn;
a battery; and
a battery antenna suitable for charging a battery via inductance charging.

In some aspects, a system is provided which can include a venous needle monitoring device including a wearable device, at least one temperature sensor, at least one heart rate sensor, a controller, and a transmitter. The system can also include a medical device including a needle, and at least one fluid line suitable for transporting fluid between the medical device and a patient, and a computer (60), a receiver (62), and transmitter (64). The at least one temperature sensor (30) and at least one heart rate sensor (40) are arranged on the wearable device (10), the controller (12) and transmitter (14) are located on or within the wearable device (10), the at least one temperature sensor (30) and at least one heart rate sensor (40) are in communication with the controller (12), the transmitter (14) transmits data to the receiver (62) of the medical device (50), and the controller (12) or computer (60) generates an alarm signal when a threshold value is reached based upon a temperature value measured by the at least one temperature sensor (30) and a heart rate value measured by the at least one heart rate sensor (40).

Such aspects may include one and/or another of (and in some aspects, a plurality of, a majority of, substantially all of, and all of) the following features, functions, functionality, structure, steps, and clarifications, leading to yet further aspects:
the data transmitted to the medical device (50) can comprise at least one temperature value measured by the at least one temperature sensor (30) and/or at least one heart rate value measured by the at least one heart rate sensor (40);
the computer can be adapted for processing the temperature and heart rate data to determine if, e.g., the threshold value has been reached;
the computer is suitable/configured for transmitting a signal to the wearable device (10) to instruct controller (12) to generate the alarm signal;
the medical device (50) is configured to generate a medical device alarm, where the medical device alarm can comprise at least one of: an audible alarm; a vibration alarm; and a visual alarm;
the medical device (50) is configured to stop transporting fluid along the fluid line (52, 54) when the threshold value is reached;
at least one associated monitoring device (90) remote from the wearable medical device (10) and the medical device (50), where the at least one associated monitoring device (90) can be in wired or wireless communication with both the wearable medical device (10) and the medical device (50);
the associated monitoring device includes a receiver for receiving the alarm signal;
the at least one associated monitoring device (90) generates an associated monitoring device alarm;
the associated medical device alarm comprises at least one of: an audible alarm; a vibration alarm; and a visual alarm;
after a threshold time is reached and/or after an alarm signal is generated, a check signal is generated by the medical device (50) computer (60) and transmitted to the at least one associated monitoring device (90); and
a charging station (100) suitable for charging a battery of the wearable device (10), where the charging station (100) can be complementary to the shape of the wearable device (10) suitable for receiving the wearable device (10), and the charging station (100) can further comprise a sensor calibrator suitable for recalibrating the sensors of the wearable device (10).

In some aspects, a method of monitoring a venous needle is provided, which can utilize the system and/or devices according to other disclosed aspects and embodiments. Such a method and includes communicating temperature sensor data and heart rate sensor data with the controller, transmitting data from the transmitter (14) to the receiver (62) of the medical device (50); and generating an alarm signal by the controller (12) or computer (60) when a threshold value of at least one of the communicated temperature sensor data and the communicated heart rate sensor data.

Such aspects may include one and/or another of (and in some aspects, a plurality of, a majority of, substantially all of, and all of) the following features, functions, functionality, structure, steps, and clarifications, leading to yet further aspects:
the data transmitted to the medical device (50) is at least one temperature value measured by the at least one temperature sensor (30) and at least one heart rate value measured by the at least one heart rate sensor (40);
a computer (60) is suitably adapted for processing the temperature and heart rate data to check if the threshold value has been reached, where the computer can be suitable for transmitting a signal to the wearable device (10) to instruct controller (12) to generate the alarm signal via transmitter (64);
stopping the transporting of fluid to or along the fluid line (52, 54) when the threshold value is reached;
a/the controller (12) further comprises a memory (18),
measuring heart rate variability using the at least heart rate sensor (40);
the threshold value is further based upon the measured heart rate variability;
providing an intra-treatment mode and an inter-treatment mode, where the intra-treatment mode can be active during a treatment session using the medical device (50), and the inter-treatment mode can be active after the treatment session is finished;
after the treatment session is completed, the wearable device enters the inter-treatment mode;
the inter-treatment mode is a low power mode; and
the heart rate and/or temperature data are obtained at a reduced frequency as compared to the intra-treatment mode.

According to a first aspect of the invention a venous needle monitoring device is described. The venous needle device comprising: a user wearable device comprising any of: an open ring, a closed ring, a band, a strap, a watch, a clip or a patch; at least one temperature sensor; at least one heart rate sensor; a controller; and a transmitter. The at least one temperature sensor and at least one heart rate sensor are arranged on the wearable device so as to be able to sense at least temperature and heart rate of the user. The controller and transmitter are located on or within the wearable device. The at least one temperature sensor and at least one heart rate sensor are in communication with the controller. The controller generates an alarm signal when a threshold value is reached based upon a temperature value measured by the at least one temperature sensor and a heart rate value measured by the at least one heart rate sensor. The transmitter is configured and arranged for transmitting the alarm signal to one or more associated medical devices.

Advantageously the wearable device allows for the measurement of temperature and heart rate values which are indicative of a needle out event.

Advantageously the wearable device raises an alarm to the user that a needle out event has occurred.

A further advantage of the user wearing the device is that it permits good skin-to-sensor contact between the user and the wearable device's sensors. Skin-to-sensor contact allows for more accurate reading of the user's vital signs.

Locating controller within the wearable electronic device means that calculations may be done within the wearable electronic device without relying on an external computer. Alternatively some computation may take place within the wearable electronic device and some on an external computer connected to the wearable electronic device.

The controller further comprises a memory and the controller is configured to measure heart rate variability using the at least heart rate sensor.

Advantageously the memory stores data from the sensors for review later (for example by a medical professional) or by a suite of data analytics software.

The ability to have the wearable electronic device as an open ring, a closed ring, a band, a strap, a watch, a clip or a patch means better skin-to-sensor contact between the wearable electronic device and the user even when the user moves.

A further advantage is that the wearable electronic device is inconspicuous and does not look like a piece of medical equipment. The user may wear the device between treatment sessions and as a result not feel conscious that they are wearing a piece of medical equipment as would be the case with other bulkier devices known in the art.

Advantageously the wearable electronic device may not look like a piece of medical equipment but rather like a piece of jewellery.

Moreover, such apparatus of the present wearable electronic device has a profile that is not conspicuous and also is not as likely to catch on pieces of clothing or medical equipment (such as a dialysis machine or medical tubing).

The threshold value is further based upon the measured heart rate variability. An advantage is the calculation and measurement of the heart rate variability over time using the wearable electronic device. Such an arrangement will advantageously allow for a further method of checking of a needle out event. Heart rate may vary over time when a user becomes stressed such as during a needle out event.

The at least one temperature sensor and at least one heart rate sensor are located on a face of the wearable electronic device.

The wearable device further comprises a vibration device suitable for generating a vibration alarm.

The vibration device advantageously allows for the user to be warned of a needle out event.

A vibration alarm is advantageous for users who may be blind (and not see a visual alarm) or deaf (and not hear an audible alarm). Further, in home treatment sessions a vibrational alarm is advantageous as it can awake a user from sleep in such a case where they may be undergoing an overnight dialysis session.

The at least one heart rate sensor comprises an infra-red heart rate sensor with at least one photodiode and at least one LED. The heart rate sensor may be a transmission type sensor. Alternatively the heart rate sensor may be a reflection type heart rate sensor. Alternatively a combination of transmission and reflection heart rate sensors may be used. Alternatively other wavelengths of light may be used in the heart rate sensor.

In one embodiment at least one photodiode and at least one LED are separate components and are disposed in separate locations on the wearable device suitably for allowing transmission photoplethysmograthy when the wearable device is worn.

In one embodiment at least one photodiode is disposed adjacent the at least one LED on the wearable electronic device suitably for allowing reflectance photoplethysmograthy when the wearable electronic device is worn.

In one embodiment the wearable electronic device further comprises a battery and battery antenna suitable for charging the battery via inductance charging.

Advantageously charging the wearable device via inductance charging means that the wearable device may be fully sealed and therefore waterproof. In such an embodiment updates to the wearable device may be made via wired connection through a connection port or wireless means using methods known in the art (e.g. Bluetooth).

The venous needle monitoring device may further comprise a blood oxygen sensor. A blood oxygen sensor is configured to measure blood oxygen saturation levels, SpO2, and/or heart rate. The blood oxygen sensor may be a transmissive pulse oximetry sensor or a reflectance pulse oximetry sensor.

The threshold value may be based upon at least one (or more of): the measured SpO2 level and/or pulse rate and/or upon a temperature value measured by the at least one temperature sensor and/or a heart rate value measured by the at least one heart rate sensor.

Advantageously, a drop in SpO2 level may indicate that a needle out event has occurred or that the needle has become dislodged. Alternatively, in some circumstances, a temporary increase in SpO2 level may indicate that a needle out event has occurred or that the needle has become dislodged.

According to a second aspect of the invention a system is described. The system comprising: a venous needle monitoring device comprising: a wearable device; at least one temperature sensor; at least one heart rate sensor; a controller and a transmitter; and a medical device comprising: a needle and at least one fluid line suitable for transporting fluid between the medical device and a patient; and a computer, a receiver and a transmitter. The at least one temperature sensor and at least one heart rate sensor are arranged on the wearable device. The controller and transmitter are located within the wearable electronic device. The at least one temperature sensor and at least one heart rate sensor are in communication with the controller. The controller transmitter transmits data to the receiver of the medical device. The controller or computer generates an alarm signal when a threshold value is reached based upon a temperature value measured by the at least one temperature sensor and a heart rate value measured by the at least one heart rate sensor.

Advantageously the wearable device allows for the measurement of temperature and heart rate values which are indicative of a needle out event.

Advantageously the wearable device or medical device raises an alarm to the user that a needle out event has occurred.

A further advantage of the user wearing the device is that it permits good skin-to-sensor contact between the user and the wearable device's sensors. Skin-to-sensor contact allows for more accurate reading of the user's vital signs.

Locating controller within the wearable device means that calculations may be done within the wearable device without relying on an external computer. Alternatively some or all of the computation may take place within the wearable electronic device and some on an external computer connected via the controller transmitter to the wearable electronic device.

In one embodiment the data transmitted to the medical device is temperature value measured by the at least one temperature sensor and heart rate values measured by the at least one heart rate sensor the computer is suitably adapted for processing the temperature and heart rate information to check if the threshold value has been reached and wherein the computer is suitable for transmitting a signal to the wearable device to instruct controller to generate the alarm signal.

In one embodiment the medical device generates a medical device alarm, wherein the medical device alarm is one or more or any combination of: an audible alarm; a vibration alarm; and/or a visual alarm.

One or more alarms can be generated to bring the needle out event to the user or medical practitioner's attention.

In one embodiment the medical device stops transporting fluid to along the fluid line when the threshold value is reached.

Advantageously stopping the transport of fluid in the medical device stops the pumping of fluid out or into the user.

In one embodiment the system further comprises at least one associated monitoring device remote from the wearable device and the medical device, and the at least one associated monitoring device is in wired or wireless communication with both the wearable device and the medical device.

In such and embodiment the associated monitoring device may be a tablet, a phone, a terminal, a computer, a monitoring station or other device. Advantageously providing a monitoring device means that the medical device and user of said medical device can be monitored remote of the medical device.

The associated monitoring device includes a receiver for receiving the alarm signal, the at least one associated monitoring device generates an associated monitoring device alarm, wherein the associated monitoring device alarm comprises at least one of: an audible alarm; a vibration alarm; and/or a visual alarm.

After a threshold time is reached after an alarm signal is generated a check signal is generated by the medical device computer and transmitted to the at least one associated monitoring devices.

The check signal is to arrange to get the attention of a medical practitioner to check on the patient.

In a further embodiment the medical device further comprises a charging station suitable for charging a battery of the wearable device, and wherein the charging station is complementary to the shape of the wearable device suitable for receiving the wearable device. The complementary shape of the charging station retains the wearable device securely reducing the chance the wearable device may be lost.

The charging station further comprises a sensor calibrator suitable for recalibrating the sensors of the wearable device. The sensor calibrator is used to recalibrate the sensors within the wearable device, this recalibration step may be undertaken whilst the wearable device is charging to reduce downtime. In alternative embodiments system updates or firmware updates may also be delivered to the wearable device at the same time as charging.

Advantageously providing a charging station on the medical device means that a patient can put the wearable electronic device back in a convenient location post treatment session. This reduces the likelihood of the wearable electronic device being lost or misplaced. Such an embodiment is useful in a dialysis ward type setting where numerous people are using the same dialysis machine in the course of a day.

The charging station is complementary to the shape of the wearable electronic device so that the charging station retains the wearable electronic device preventing it from falling off easily.

Alternatively multiple charging stations may be provided for multiple wearable electronic devices. For example if the wearable electronic device is a ring then a “small”, “medium” and “large” ring may be provided with an associated sized charging station. Providing multiple sized wearable electronic devices is advantageous as it means that different people with different sized fingers can wear a ring sized to fit them. This is advantageous in a ward type setting or dialysis clinic where multiple people may be using the same dialysis machine over the course of a day. Providing multiple rings reduces downtime as the user can choose one of the rings which fits one of their fingers that has the most charge.

Alternatively the ring can be worn on the most comfortable finger. Or on a finger that the user does not already have a ring on. Said finger may be different in size to their ring finger which may already have a wedding band on.

A third aspect of the invention is a method of monitoring a venous needle. The method comprises: providing a system comprising a venous needle monitoring device comprising: a wearable device; at least one temperature sensor; at least one heart rate sensor; a controller and a transmitter; and a medical device comprising: needle and a fluid line suitable for transporting fluid between the medical device and a patient; and a computer, a receiver and a transmitter; wherein the at least one temperature sensor and at least one heart rate sensor are disposed on the wearable device; the controller and transmitter are located within the wearable electronic device; communicating temperature sensor data and heart rate sensor data with the controller; transmitting data from the transmitter to the receiver of the medical device; generating an alarm signal by the controller or computer when a threshold value is reached based the communicated temperature sensor data and the communicated heart rate sensor data.

Further, the data transmitted to the medical device is at least one temperature value measured by the at least one temperature sensor and at least one heart rate value measured by the at least one heart rate sensor;
the computer is suitably adapted for processing the temperature and heart rate data to check if the threshold value has been reached;
and wherein the computer is suitable for transmitting a signal to the wearable electronic device to instruct controller to generate the alarm signal via transmitter.

Further, the method comprises the step of the medical device stopping transporting fluid to along the fluid line when the threshold value is reached.

In one embodiment the controller further comprises a memory and/or the method further comprises measuring heart rate variability using the at least heart rate sensor.

The threshold value is further based upon the measured heart rate variability.

In one embodiment the wearable device has an intra-treatment mode and an inter-treatment mode; wherein the intra-treatment mode is active during a treatment session using the medical device and wherein the inter-treatment mode is active after the treatment session is finished.

Further, after the treatment session using medical device is completed the wearable device enters the inter-treatment mode; wherein the inter-treatment mode is a low power mode; preferably wherein the heart rate and temperature data are obtained at a reduced frequency as compared to the intra-treatment mode.

### BRIEF DESCRIPTION OF THE DRAWINGS

One or more embodiments of the invention will now be described, by way of example only, with reference to the accompanying drawings.

FIG. 1a is a wearable electronic device for monitoring venous needle dislodgement.

FIG. 1b is a inductive charging station for the wearable electronic device of FIG. 1a.

FIG. 2 is a system including the wearable electronic device of FIG. 1a and a medical device.

FIG. 3 is an alternative embodiment of the wearable electronic device of FIG. 1a.

FIG. 4 is an alternative embodiment of the wearable electronic device of FIG. 1a.

FIG. 5 is a further embodiment of the system in FIG. 2, wherein the system further comprises a remote monitoring device.

FIG. 6 is a flowchart describing a method of monitoring for venous needle dislodgement.

FIG. 7 is a further embodiment describing a method of monitoring for venous needle dislodgement.

### DETAILED DESCRIPTION

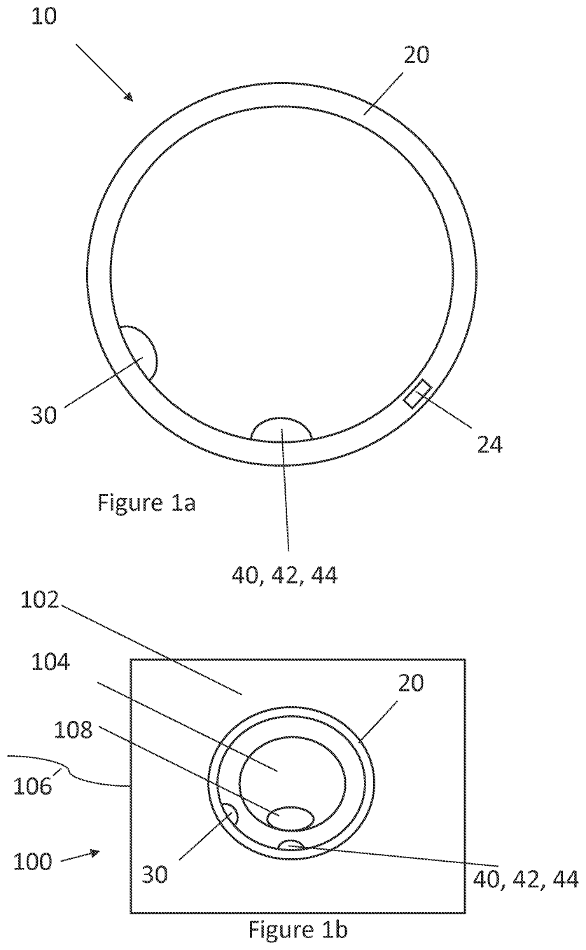

The present invention relates to a venous needle dislodgement device comprising a wearable electronic device 10 shown in FIG. 1a and FIG. 1b, at least one temperature sensor 30, at least one heart rate sensor 40, and a controller 12 with a transmitter 14.

As shown in FIG. 1a the wearable electronic device 10 is a ring with a band 20. In alternative embodiments the wearable electronic device may be an open ring, closed ring, a band, a strap, a watch, a watch strap, a clip or patch. The wearable electronic device 10 may be any other wearable device known in the art. The wearable electronic device 10 may be designed as a piece of jewellery such that it may not appear to be a piece of medical apparatus at first glance.

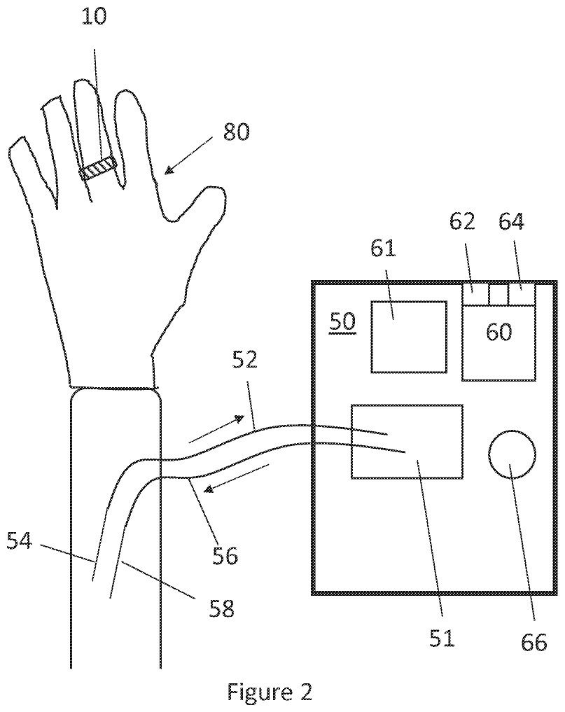

A wearable electronic device 10 is shown in FIG. 1a and in FIG. 2 being worn on a person's 80 finger. The wearable electronic device 10 in the embodiment shown in FIG. 1 is sized to fit around a person's 80 finger snuggly to prevent said wearable electronic device 10 from falling off a user's finger. The advantage is that if the person 80 wishes to they may continually wear the wearable electronic device 10 without the need to remove it between treatment sessions. Keeping the wearable electronic device on a person's 80 during finger in between treatment means it is less likely to be lost or misplaced.

Returning to FIG. 1a, the temperature sensor(s) 30 and heart sensor(s) 40 are shown disposed on or in the wearable electronic device 10. In the present embodiment the wearable electronic device 10 has a single temperature sensor 30 and a single heart sensor 40. In alternative embodiments alternative numbers of sensors 30, 40 are present. The sensors shown in FIG. 1a are shown extending from the ring 20 for the purpose of illustration and of teaching the invention, in actuality the sensors 30, 40 may be flush with the surface of the ring 20 or extend outwards to a greater or smaller degree, or conversely they may be recessed from the surface of the ring 20.

Preferably the temperature sensor 30 is located on an inner face of the band 20 such that the temperature sensor 30 faces the centre of ring 20. Preferably the heart sensor 40 is located on an inner face of the band 20 such that the heart sensor 40 faces the centre of ring 20.

The temperature sensor 30 is a negative temperature coefficient thermistor. In alternative embodiments the temperature sensor may be any temperature sensor known to the skilled person. The temperature sensor 30 is located on an inner face of the wearable electronic device 10. Locating the temperature sensor 30 on the inner face of the wearable electronic device 10 facilitates good contact with a person's skin such for a better temperature reading.

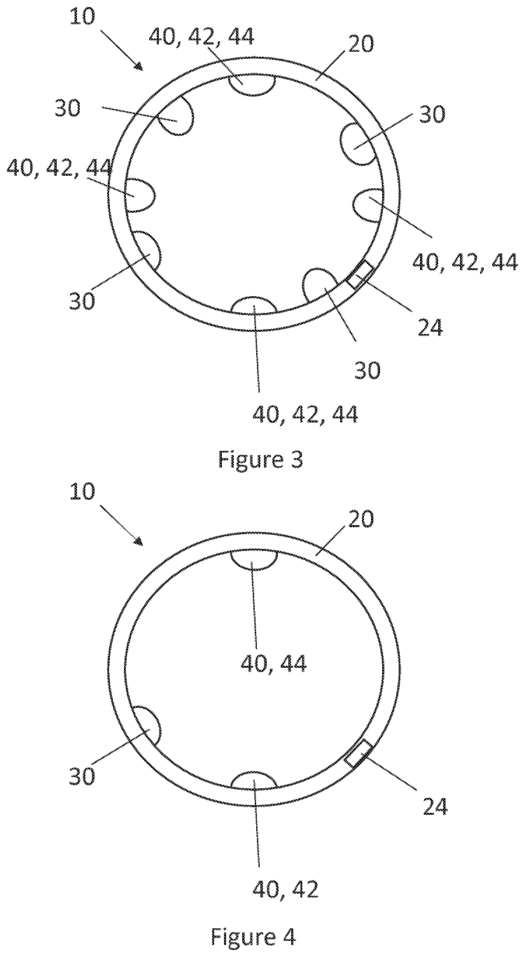

In alternative embodiments a plurality of temperature sensors 30 can be disposed in or on the wearable electronic device 10 as shown in FIG. 3. The number of temperature sensors 30 in the alternative embodiment can be at least 1-10 or at least 1-5 or at least 2 or at least 3 or at least 4 or at least 5. In the alternative embodiments the plurality of temperature sensors 30 can be disposed circumferentially around the wearable electronic device 10. The plurality of temperature sensors 30 may be evenly spaced or unevenly spaced around the circumference of the band 20. Having multiple temperature sensors 30 allows for a more accurate temperature measurement by sampling a more general volume than a single temperature sensor 30.

The heart rate sensor 40 is used to measure heart rate and also to measure heart rate variability over time.

The heart rate sensor 40 is located on the inner face of the wearable electronic device 10. The heart rate sensor 40 is an infra-red sensor with a photodiode 42 and LED 44. Preferably the photodiode 42 is disposed adjacent the infra-red LED 42. Such that the photodiode 42 and LED 44 permit reflectance photoplethysmograthy as in FIG. 1a. In alternative embodiments the at least one photodiode 42 and at least one infra-red LED 44 are separate and disposed in separate locations on the wearable electronic device 10. Such as shown in FIG. 4. Disposing the photodiode 42 and LED 44 separately on the wearable electronic device allows for transmission photoplethysmograthy.

In alternative embodiments a plurality of heart rate sensor 40 can be disposed in or on the wearable electronic device 10 as shown in FIG. 3. In the embodiment shown in FIG. 3 the heart rate sensors 40 may be transmission or reflectance heart rate sensors 40. The plurality of heart rate sensors 40 may be used to obtain a more accurate hear rate measurement.

The number of heart rate sensors 40 in the alternative embodiment of FIG. 3 can be at least 1-100 or at least 1-5 or at least 2 or at least 3 or at least 4 or at least 5. In the alternative embodiments the plurality of heart rate sensor 40 can be disposed circumferentially around the wearable electronic device 10. The plurality of heart rate sensors 40 may be evenly spaced or unevenly spaced around the circumference of the band 20.

In alternative embodiments alternative heart rate sensors may be used known in the art for example an electrical heart-rate monitor that may be used to obtain an electrocardiogram.

The venous needle monitoring device may further comprise a blood oxygen sensor. The blood oxygen sensor is configured to measure blood oxygen saturation levels, SpO2, and/or heart rate. The blood oxygen sensor may be a transmissive pulse oximetry sensor or a reflectance pulse oximetry sensor. The blood oxygen sensor may be disposed on the wearable electronic device 10 in a similar fashion to the heart rate sensor 40 or temperature sensor 30. The blood oxygen sensor is in communication with the controller 12 in a similar fashion as the heart rate sensor 40 or temperature sensor 30.

The controller 12 and transmitter 14 are located within the wearable electronic device 10. Said controller 12 and transmitter 14 are in electronic communication with the temperature sensor 30 and heart rate sensor 40 via means known to the skilled person, for example via wired electronic communication.

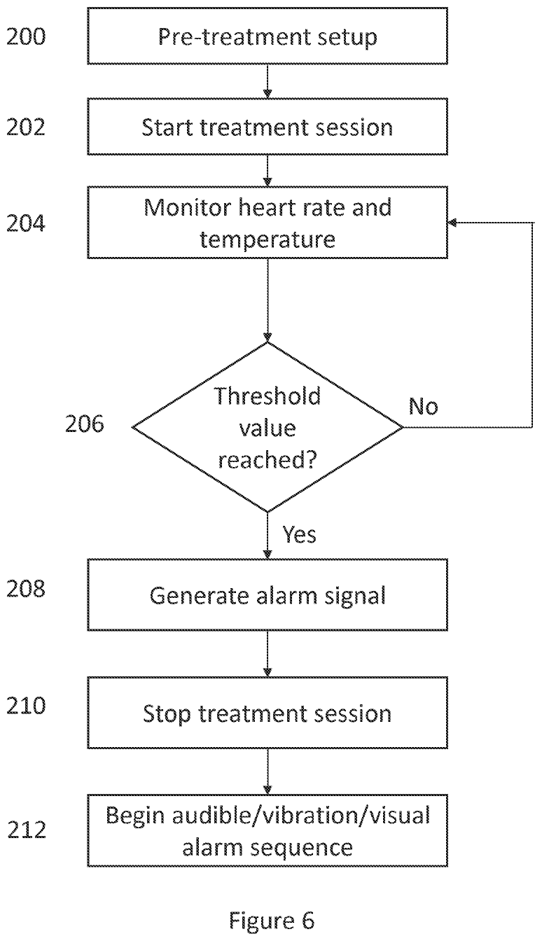

The wearable electronic device 10 has an intra-treatment mode and an inter-treatment mode. The intra-treatment mode is used during a treatment session. Outside of a treatment session (and the intra-treatment mode) the wearable electronic device 10 enters an inter-treatment mode. The intra-treatment mode will be described in more detail below with respect to FIG. 6.

The inter-treatment mode is a “normal” (non-treatment) mode for the wearable electronic device, in the inter-treatment mode the data collection of heart rate and temperature data may be reduced to conserve battery for the next treatment session (i.e. a low power mode). Optionally the inter-treatment mode may obtain baseline heart rate and temperature data used to track the health of the user 80. Optionally further, the baseline data may be incorporated into an algorithm for judging if a needle out event has occurred.

The wearable electronic device 10 has a battery used to provide power to the wearable electronic device. Preferably the battery has a battery antenna suitable for charging the battery via inductance charging using the charging station 100 shown in FIG. 1b. Advantageously charging using inductance charging means that the wearable electronic device can be a sealed component which may be splash proof or water proof. In alternative embodiments the battery may be charged using a wired connection with charging being achieved by means of a charging port (such as a USB-C port) and associated charging cable.

The optional charging station 100 will be described in detail with reference to FIG. 1b. The charging station 100 comprises: a base 102, a power transmitter tower 104, a sensor calibrator 108 and a power wire 106.

The charging station 100 power transmitter tower 104 is located on the base 102. The power transmitter tower 104 contains a wireless power transmission coil within the power transmitter 104. The power transmitter tower 104 is connected to the power wire 106. The power wire 106 is connectable to a mains electricity source or a battery source of electricity using known means.

The power transmitter tower 104 is sized to fit within the band 20 when the band 20 is placed on the charging station 100 as shown in FIG. 1a. To reduce power losses when transmitting power wirelessly the distance between the inner face of band 20 and the power transmission tower 104 is minimized as much as possible such that there is a tight fit of the band 20 around power transmission tower 104. However, not so tight a fit that it does not enable the band 20 to be removed from the charging station 100.

In alternative embodiments the band 20 may have a projection that is complementary to a groove in base 102 to ensure correct alignment of the band 20 with the charging station 100 and optimal charging.

The sensor calibrator 108 is used to calibrate the heart rate sensor 40 when the device is placed on the charging station. The sensor calibrator 108 is used to check whether the heart rate sensor 40 is calibrated correctly or whether a recalibration of the wearable electronic device is required. The sensor calibrator 108 may be a master crystal clock suitable for calibrating the heart rate sensor 40.

In alternative embodiments where transmission photoplethysmograthy is used for calculating heart rate in the wearable electronic device 10 then the power transmitter tower 104 may be transparent to the wavelength of light used in the sensor to permit calibration. In this embodiment the sensor calibrator 108 may be a master crystal clock suitable for calibrating the heart rate sensor 40.

In further embodiments the sensor calibrator 108 may further include a temperature calibrator for checking the calibration of temperature sensor 30.

In further embodiments the charging station 100 may have a charge indicator. The charge indicator may be a light emitting diode (LED) which changes colour dependent upon the charge in the battery.

The wearable electronic device 10 may further comprise a vibration device 24 suitable for generating a vibration alarm. The vibration device 24 is located within the wearable electronic device 10. Preferably the vibration device is located within band 20 of the wearable electronic device 10. Preferably the vibration device 24 is a piezoelectric device or micro electric mechanical system. More preferably the vibration device 24 is a micro electric mechanical system.

With the aid of FIGS. 1 and 2 the system for detecting venous needle dislodgement will herein be described. The system incorporates the wearable electronic device 10 as described above and shown worn on a person 80 in FIG. 2 and a medical device 50 also shown in FIG. 2. The medical device 50 comprises a needle 54, 58, at least one fluid line 52, 56, a computer 60, a receiver 62 and a transmitter 64.

The medical device 50 is a fluid dispensing medical device and preferably a dialysis machine. An example of a dialysis machine is known from the applicant's international application WO2019002837A1.

A brief synopsis of the applicant's international application WO2019002837A1 the description of which is herewith incorporated by reference (and especially incorporated is the description of WO2019002837A1 FIG. 1 which describes and shows a schematic of a dialysis system having a disposable cartridge comprising a fluid path defined by pumps and valves) describes how the dialysis machine 50 (of present FIG. 2) is used to remove contaminants from the blood of a patient. Fluid is removed from a patient 80 using a needle 54 and line 52. The fluid is cleaned in fluid cleaning circuit 51 where the fluid is filtered. The clean fluid is then sent back to the patient via line 56 and needle 58.

In alternative embodiments the medical device may be any one of: an artificial heart pump, a blood pump, an infusion device, an infusion pump, or other such medical device known in the art which is used to remove fluid from a patient and/or inject fluid into a patient or remove fluid and replace fluid into a patient using a needle.

The medical device 50 may be located on a ward, such as a renal ward, or in a patient's home allowing for home use of the medical device 50.

The medical device computer 60 has a receiver 62 and transmitter 64 that is in wireless communication with the wearable electronic device 10. Said communication may be by means of Bluetooth, Wi-Fi, infra-red, or other suitable means known to the art.

The medical device computer 60 has a CPU, memory and an input means for allowing user inputs. Preferably the input means is a touch screen device 61 with a GUI. The GUI has buttons for controlling the medical device 50. In alternative embodiments the medical device 50 is controllable using buttons which are disposed on the casing of the medical device.

The medical device 50 may further comprise a speaker 66. The speaker 66 is suitable for transmitting an audible alarm.

The medical device 50 may further comprise the charging station 100. In such an embodiment the charging station 100 may be disposed on the housing of the dialysis machine.

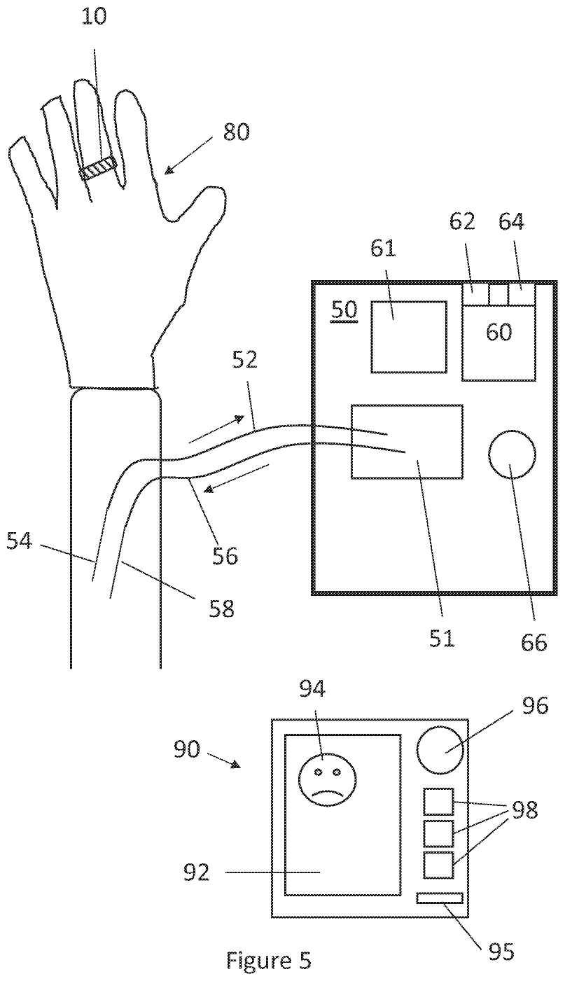

A further embodiment of the system is shown in FIG. 5 and will herein be described.

The system of FIG. 5 further comprises at least one associated monitoring device 90. The associated monitoring device 90 comprises a portable computing device with a display 92, a speaker 96, a vibration device 95, and buttons 98 for controlling the associated monitoring device 80.

The associated monitoring device 90 is in wired or wireless communication with: both the wearable electronic device 10 and the medical device 50; or solely the wearable electronic device 10; or solely the medical device 50. The wired or wireless communication is achieved via means as described above with reference to the connection between the wearable electronic device 10 and medical device 50 or through alternative means known in the art.

The associated monitoring device 90 is remote from the medical device 50.

The vibration device 95 is located within the associated monitoring device 90. Preferably the vibration device 95 is a piezoelectric device or micro electric mechanical system. More preferably the vibration device 95 is a micro electric mechanical system.

The speaker 96 is disposed on or in the housing of the associated monitoring device 90. The speaker is suitable for emitting an audible alarm.

The display 92 is disposed on or in the housing of the associated monitoring device 90. The display 92 may flash or display a warning symbol when an alert signal is generated. The warning symbol advantageously draws attention to a user of the associated monitoring device 90.

In alternative embodiments no buttons 98 are provided on the associated monitoring device 90 for use in controlling the device (however an on/off means and volume means may be provided using buttons). Instead the associated monitoring device 90 may be controlled using a GUI displayed on display 92.

The associated medical device 90 may be a phone or tablet. In such embodiments the phone or tablet device may be an “off-the-shelf” device with monitoring software either pre-installed or installed onto the device enabling communication with the wearable electronic device 10 and the medical device 50.

In such an embodiment where the medical device 90 is a phone or tablet that has a camera with a flash bulb or light emitting diode (LED) the bulb or may additionally be used as a visual warning. The visual warning in this instance being undertaken by the flashing on and off of the bulb or LED. The frequency of the flashing may be predetermined.

In further embodiments the associated medical device 90 may be a terminal, computer or monitoring station. The terminal, computer or monitoring station may be located in a ward or renal ward or dialysis centre, or other location where medical procedures involving a needle 54, 58 is intended to be undertaken by one or more patients simultaneously using different medical devices 50. An example is in a dialysis centre wherein a number of persons 80 undertake individual dialysis sessions using individual dialysis machines 50.

In such an embodiment a number of different dialysis machines 50 or medical devices 50 may be connected to the associated monitoring device 50 and/or the wearable electronic devices 10. The single “station” monitoring all the medical devices 50 and wearable electronic devices 10 advantageously allows for simultaneous monitoring of all the individual medical treatments being carried out. In such an embodiment the associated monitoring device 90 may further be adapted to control and instruct any cleaning operations to the medical device 50 when a treatment session has finished.

In alternative embodiments a number of different associated medical devices 90 may be in communication with the medical device 50 and/or the wearable electronic device. For example the monitoring station and multiple phones, tablets, and/or terminals may be connected at the same time forming a monitoring system.

With the aid of FIGS. 1, 2, 5 and 6 a method for checking for venous needle dislodgement will be described.

Pre-Treatment Setup

Before a treatment session is started a pre-treatment setup 200 is initiated the wearable electronic device 10 is paired with the medical device 50, such as via a Bluetooth handshake or other means known in the art. The user 80 can pair the device using set up instructions displayed on the medical device screen 61. This pairing may need only be done once before the first in a series of treatment sessions using the same wearable electronic device 10 and medical device 50 pairing. The wearable electronic device 10 and medical device 50 will “remember” that they have been previously paired so as not having to go through a further pairing step at the start of a subsequent session saving time for the user.

Before the session starts the wearable electronic device 10 enters intra-treatment mode. Such a mode may be entered automatically when the user 80 or practitioner begins setting up the apparatus for a session or the intra-treatment mode may be manually entered once the setup has been completed by selecting a button on the medical device 50.

Once the intra-treatment mode has been entered the wearable electronic device 10 transmits heart rate and temperature data to the paired medical device 50. In other embodiments the wearable electronic device may transmit heart rate and temperature data before entering the intra-treatment mode and during the inter-treatment mode after the wearable electronic device has been paired.

The user will insert (or have inserted for them) needle 54 into their arm which is connected to the withdrawal line 52 and fluid circuit 51 of dialysis machine 50.

The user will insert (or have inserted for them) needle 56 into their arm which is connected to the return line 52 and fluid circuit 51 of dialysis machine 50.

The dialysis machine 50 may request that the user 80 or a medical practitioner inserting the needles 52, 54 into user 80 to confirm that the needles 52, 54 have been inserted into the arm of person 80. The dialysis machine may also request that the wearable electronic device 10 is correctly in place on user 80. In this embodiment the treatment session will not begin until the user 80 or medical practitioner has confirmed both needles are correctly inserted. In further embodiments, the treatment session may not begin until the medical device 50 confirms it is receiving heart rate and temperature data from the wearable electronic device 10.

Optionally the medical device 50 may confirm that the needles 54, 58 and lines 52, 56 are in place by priming the fluid circuit 51 with fluid withdrawn from the user 80 using pumps in the fluid circuit 51. If the fluid circuit 51 may check for fluid withdrawal using known means. If the medical device 50 does not withdraw fluid from the user 80 then an error may be displayed and pre-treatment may be stopped until the error in needle 54, 58 or line 52, 56 is corrected.

Treatment Session

A treatment session 202 is then begun. During the treatment session 202 fluid is withdrawn via needle 54 and line 52 from the patient and pumped in to the dialysis machine 50. The fluid is then treated and returned to the patient via the return line 56 and needle 58.

Continuously during a treatment session step 204 the wearable electronic device 10 monitors the heart rate and temperature of the patient using the temperature sensor 30 and heart rate sensor 40 respectively to check if a condition 206 (a threshold value) has been reached. The condition 206 is indicative of a needle out event.

The heart rate and temperature information is then processed either using controller 12 in the wearable electronic device or computer 60 of the dialysis machine 50 to check whether a threshold value is reached.

The threshold value 206 is based on: heart rate and temperature of person 80. In a further embodiment the threshold value is also based upon the heart rate variability measured over a period of time. The heart rate variability may be calculated using methods known in the art.

Where a blood oxygen sensor is provided in the wearable electronic device 10 the threshold value may be further based upon oxygen saturation. This may be based upon a deviation of blood oxygen saturation levels over a period of time. The device may establish a user baseline during or before the start of treatment related to a set period of time. The monitoring may consist of detecting a variation from that baseline. The variation may be a 3 to 4% deviation from the baseline. The deviation may be positive or negative.

In an alternative embodiment the heart rate and temperature information is then processed either using controller 12 in the wearable electronic device or computer 60 of the dialysis machine 50 to check whether an alarm condition is reached in a monitoring algorithm.

The alarm condition may be based on: heart rate and temperature of user 80. In a further embodiment the threshold value is also based upon the heart rate variability. For example the alarm condition may be based on an increased heart rate variability measured over a number of seconds which deviates from allowed normal values indicative of a normal treatment session.

If the threshold value is reached then an alarm signal is generated in step 208 and sent to the wearable electronic device 10, the medical device 50 and if any associated medical devices 90 are connected to the associated medical devices 90 also.

Optionally there may be a delay before the alarm signal 208 is generated. The delay may be 1-10 seconds 1-5 seconds or 1 or 2 or 3 or 4 or 5 seconds. The delay may be used to confirm that the threshold value 306 has been reached and may be used to prevent false positive alarm signals being generated.

The alarm signal alerts the medical device 50 to stop treatment at step 210. The stopping of the treatment at step 210 may involve shutting off any pumps used to remove or return fluid to a user 80. In further embodiments the alarm signal may indicate the medical device 50 to enter a “failsafe” mode if such a mode is available to the medical device 50 which the wearable electronic device 10 is paired to.

When the treatment is stopped the audible, vibration, visual alarm begins at step 212. The alarms are to alert the patient that a needle out event is likely to have occurred. The audible alarm is sounded using speaker 66. The alarm is loud to alert the person 80 and any person in the vicinity to person 80 to the fact a needle may have fallen out and that person 80 must be checked on.

The audible alarm is also sounded using the speakers of any associated medical devices 90, such as speaker 96.

The visual alarm is displayed on the dialysis machine 50 display 61. The visual alarm 94 may take the form of a graphical warning (as shown in FIG. 5 on associated medical device 90). The visual alarm 94 may also be a flashing light whereby the display 61 flashes one or more different colours to alert the person 80. In a further embodiment the medical device 50 may have an alarm light which flashes brightly in order to catch the attention of the person 80 when the alarm signal is generated.

The visual alarm may also displayed on any associated medical devices 90 at the same time as the medical device 50.

The vibration alarm is made by the vibration device 24 in the wearable electronic device 10. The vibration alarm vibrates the wearable electronic device to alert the wearer that there has been a potential needle out event occurring.

The vibration alarm may also be made by the associated medical device 90 using the vibration device 95.

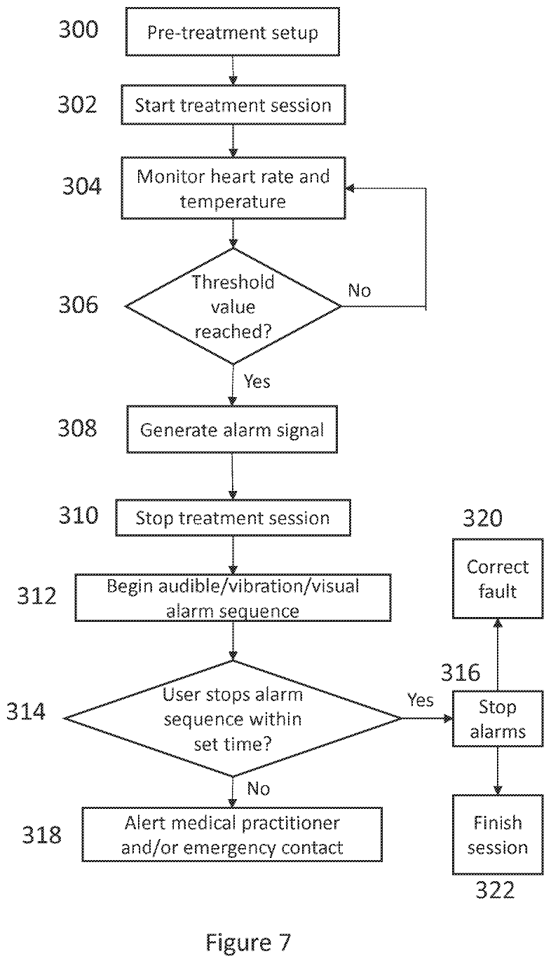

With the aid of FIGS. 1, 2, 5 and 7 a method for checking for venous needle dislodgement will be described. The features of FIG. 7 which are common with FIG. 6 are prefixed by a “3”. Steps 300 to 312 are identical to steps 200 to 212 of FIG. 6 and the method described above.

In the embodiment of FIG. 7 once the system begins the audible/vibration/visual alarm sequence of step 312 a further step 314 is begun.

Step 314 is an optional step whereby the user 80 or a medical practitioner (such as a treatment supervisor) can stop the alarm sequence within a set time period to a) continue the treatment session if the alarm was raised in error (e.g. if the user 80 removes the wearable electronic device 10 so no heart rate is detected) or b) provide assistance to the user 80.

Step 314 is achieved in two parts:

1) The user 80 or medical supervisor is alerted to the alarm during the set time period wherein the time period to check on the user 80 may be 0-360 seconds, 0-180 seconds, 0-60 seconds, 0-30 seconds, or 30 seconds from the start of step 312.

The alarm may then be stopped at step 316 before attending to the patient by entering a password into the display 61 of the medical device 50, scanning a QR code displayed on display 61 using a camera of an associated medical device, or selecting a stop alarm button located on the medical device 50.

Optionally the information on who stopped the alarm and a time and date stamp for this operation may be saved in the medical device 50 memory.

2) The alarm sequence may then be stopped by correcting the fault at step 320 (e.g. replacing ring on finger if removed or replacing needle 52, 54 if it has slipped out of position or replacing the needle 54, 58 and fluid line 52, 56 itself), then once the system reobtains heart rate and temperature information from the patient that is below the threshold value the user 80 or supervisor of the treatment session can set the treatment to restart or choose to finish the treatment session early at step 322.

Optionally the treatment session may only be restarted at step 320 by the user 80 or supervisor entering a password into the display 61 of the medical device 50, scanning a QR code displayed on display 61 using a camera of an associated medical device, or selecting a restart button located on the medical device 50. Optionally the information on who restated the session and a time and date stamp for this operation may be saved in the medical device 50 memory.

If the user 80 or supervisor does not stop the alarm within the set time period then a further alert (a check signal) is sent to a medical practitioner(s) and/or emergency contact to check on the patient.

In a hospital or ward setting such a check signal may be implemented as part of a continuous non-invasive ward monitoring system whereby alerts are sent to one or more medical practitioners simultaneously via a paging, telephone, text, or electronic messaging system instructing the medical practitioner(s) to check on the user 80. Said alert may contain information as to which medical device the user 80 is using and/or where to find the patient or patient information.

In a setting where the medical device 50 is being used in the user's 80 home then they may be the sole person present at the address. As a result if the user becomes in some way incapacitated (e.g. asleep or unconscious), during a dialysis session for example, they may be unable to correct the fault which began the alarm sequence 312. In such a situation the further alert signal may be sent to a medical practitioner or call centre operative or an emergency contact who may then attempt to get in contact with the user 80 and check that they are ok. In such a situation where contact cannot be reached with the user 80 the medical practitioner, call centre operative or emergency contact may further optionally inform emergency services to check on the user 80.

While various inventive embodiments have been described and illustrated herein, those of ordinary skill in the art will readily envision a variety of other means, steps, and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant only to be examples and that actual parameters, dimensions, materials, and configurations will depend upon the specific application or applications for which the inventive teachings is/are used. Those skilled in the art will also recognize, or be able to ascertain using no more than routine experimentation, many equivalents to the specific inventive embodiments described herein. It is, therefore, to be understood that the foregoing disclosed embodiments are presented by way of example only and that, within the scope of claims supported by the present disclosure (including equivalents thereto), inventive embodiments may be practiced otherwise than as specifically described and claimed.

Some of the inventive embodiments of the present disclosure are directed to each individual feature, system, article, material, kit, method, and step, described herein. In addition, any combination of two or more such features, systems, articles, materials, kits, methods, and steps, if such features, systems, articles, materials, kits, methods, and steps, are not mutually inconsistent, is included within the inventive scope of the present disclosure. Some embodiments disclosed herein may also be combined with one or more features, as well as complete systems, devices or methods of other embodiments (as well as known systems, devices, or methods) to yield yet other embodiments and inventions. Moreover, some embodiments, may be distinguishable from the prior art by specifically lacking one and/or another feature disclosed in the particular prior art reference(s); i.e., claims to some embodiments may be distinguishable from the prior art by including one or more negative limitations.

Also, as shown above, various inventive concepts may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Any and all references to publications or other documents, including but not limited to, patents, patent applications, articles, webpages, books, etc., presented anywhere in the present application, are herein incorporated by reference in their entirety. Moreover, all definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The terms “can” and “may” are used interchangeably in the present disclosure, and indicate that the referred to element, component, structure, function, functionality, objective, advantage, operation, step, process, apparatus, system, device, result, or clarification, has the ability to be used, included, or produced, or otherwise stand for the proposition indicated in the statement for which the term is used (or referred to) for a particular embodiment(s).

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases.

What is claimed is:

1. A venous needle monitoring device comprising:
a wearable device;
at least one temperature sensor arranged on the wearable device to sense temperature of a user wearing the wearable device;
at least one heart rate sensor arranged on the wearable device to sense heart rate of the user wearing the wearable device;
a controller carried on the wearable device, the controller in communication with the at least one temperature sensor and the at least one heart rate sensor, the controller configured to generate an alarm signal when a threshold value is reached based upon a temperature value measured by the at least one temperature sensor and a heart rate value measured by the at least one heart rate sensor; and
a transmitter carried on the wearable device, the transmitter configured to transmit the alarm signal to one or more medical devices treating the user wearing the wearable device.

2. The venous needle monitoring device of claim 1, wherein the controller is further configured to measure heart rate variability using the at least one heart rate sensor.

3. The venous needle monitoring device of claim 2, wherein the threshold value is further based upon heart rate variability measured using the at least one heart rate sensor.

4. The venous needle monitoring device of claim 1, wherein the at least one temperature sensor and at least one heart rate sensor are located on a face of the wearable device.

5. The venous needle monitoring device of claim 1, further comprising at least one blood oxygen sensor, wherein the at least one blood oxygen sensor is arranged on the wearable device to sense blood oxygen saturation levels, SpO2, and/or heart rate of the user, and wherein the blood oxygen sensor is in communication with the controller.

6. The venous needle monitoring device of claim 5 where the blood oxygen sensor is at least one of a transmissive pulse oximetry sensor or a reflectance pulse oximetry sensor.

7. The venous needle monitoring device of claim 1, wherein the wearable device further comprises a vibration device suitable for generating a vibration alarm.

8. The venous needle monitoring device of claim 1, wherein the at least one heart rate sensor comprises an infra-red heart rate sensor including at least one photodiode and at least one LED.

9. The venous needle monitoring device of claim 8, wherein the at least one photodiode and at least one LED are spatially separated from one another on the wearable device for transmission photoplethysmograthy when the wearable device is worn by the user.

10. The venous needle monitoring device of claim 8 wherein the at least one photodiode is disposed adjacent the at least one LED on the wearable device for reflectance photoplethysmograthy when the wearable device is worn by the user.

11. The venous needle monitoring device of claim 1, wherein the wearable device further comprises a battery and a battery antenna, wherein the battery is chargeable through inductance charging via the battery antenna.

12. A system comprising:
a medical device comprising:
a needle and at least one fluid line through which fluid is transportable between a patient and the medical device,
a receiver,
a first transmitter, and
a computer in communication with a receiver and a first transmitter; and
a venous needle monitoring device comprising
at least one temperature sensor,
at least one heart rate sensor,
a controller in communication with the at least one temperature sensor and the at least one heart rate sensor,
a second transmitter operable to transmit data to the receiver of the medical device, and
a wearable device on which the at least one temperature sensor, the at least one heart rate sensor, the controller, and the second transmitter are arranged,
wherein at least one of the controller or the computer is configured to generate an alarm signal when a threshold value is reached based upon a temperature value measured by the at least one temperature sensor on the wearable device and a heart rate value measured by the at least one heart rate sensor on the wearable device.

13. The system of claim 12, wherein the controller is configured to transmit to the computer, via the second transmitter, at least one temperature value measured by the at least one temperature sensor and at least one heart rate value measured by the at least one heart rate sensor, the computer is configured to process the at least one temperature value and the at least one heart rate value to determine if the threshold value has been reached and to transmit to the controller, via the second transmitter, a signal to instruct the controller to generate the alarm signal.

14. The system of claim 12, wherein the medical device is configured to generate a medical device alarm, wherein the medical device alarm comprises at least one of an audible alarm, a vibration alarm, or a visual alarm.

15. The system of claim 12, wherein the medical device is configured to stop transporting fluid along the fluid line when the threshold value is reached.

16. The system of claim 12, further comprising at least one monitoring device remote from each one of the wearable device and the medical device, wherein the at least one monitoring device is in wired or wireless communication with both the wearable device and the medical device.

17. The system of claim 12, wherein the medical device further comprises a charging station in which a battery of the wearable device is chargeable, wherein the charging station is complementary to the shape of the wearable device suitable for receiving the wearable device.

18. The system of claim 17, wherein the charging station further comprises a sensor calibrator configured to calibrate the at least one temperature sensor and the at least one heart rate sensor.

19. A method of monitoring a venous needle comprising:
providing a system comprising
a medical device comprising
a needle and at least one fluid line through which fluid is transportable between a patient and the medical device,
a receiver,
a first transmitter, and
a computer in communication with a receiver and a first transmitter; and
a venous needle monitoring device comprising
at least one temperature sensor,
at least one heart rate sensor,
a controller in communication with the at least one temperature sensor and the at least one heart rate sensor,
a second transmitter operable to transmit data to the receiver of the medical device, and
a wearable device on which the at least one temperature sensor, the at least one heart rate sensor, the controller, and the second transmitter are arranged;
transmitting data from the at least one temperature sensor and the at least one heart rate sensor from the second transmitter to the receiver; and
generating an alarm by the controller or the computer based on a threshold value of the data from the at least one temperature sensor and the at least one heart rate sensor.

20. The method of claim 19, further comprising stopping transport of fluid to or along the fluid line when the threshold value of the data is reached.
